(19) United States
(12) Patent Application Publication
(10) Pub. No.: US 2019/0029664 A1
(43) Pub. Date: Jan. 31, 2019

(54) SUTURE ANCHOR WITH DEFORMABLE CAP

(71) Applicant: Smith & Nephew, Inc., Memphis, TN (US)

(72) Inventors: Richard M. Lunn, Memphis, TN (US); Timothy Young, Memphis, TN (US)

(73) Assignee: Smith & Nephew, Inc., Memphis, TN (US)

(21) Appl. No.: 16/074,861

(22) PCT Filed: Feb. 10, 2017
(86) PCT No.: PCT/US2017/017370

§ 371 Date: Aug. 2, 2018

Related U.S. Application Data
(60) Provisional application No. 62/294,391, filed on Feb. 12, 2016.

(51) Int. Cl. A61B 17/04

(52) U.S. Cl. CPC A61B 17/0401; A61B 2017/0445; A61B 2017/00867; A61B 2017/0438; A61B 2017/0424

(57) ABSTRACT

An expanding suture anchor in which a distal end of the rigid anchor body includes at least one annular protrusion. A mating deformable cap includes at least one annular slot or ridge that engages the annular protrusion such that the annular slot and annular protrusion are coupled when the deformable cap is pushed or pulled onto the rigid anchor body. The engagement of the annular slot and the annular protrusion may lock the deformable cap and rigid anchor body into a mated position and inhibits distal retraction of the deformable cap from the anchor body.

### TECHNICAL FIELD

This present disclosure relates to a suture anchor for a surgical procedure and, more particularly, to an expanding suture anchor with a deformable cap.

### BACKGROUND

Surgeons use a variety of suture anchors during soft tissue repair. For example, a suture anchor may be secured into bone through an interference fit with a pre-drilled bone hole. Maximum interference is desirable as resulting in higher bone fixation strength and less chance of the suture anchor pulling out of bone. While maximum interference is desirable and easy to obtain with larger diameter anchors, suture anchors in general are decreasing in size which allows surgeons to place more anchors in the same amount of area and create a more precise repair. As suture anchors decrease in diameter, however, their column strength also decreases. Lower column strength makes it more difficult to insert small diameter suture anchors into bone, especially when the bone is dense or the anchor insertion trajectory varies from the originally drilled bone trajectory due to small operator movements during usage. Because small diameter suture anchors are less structurally sound and require greater force to insert them, there is an increased risk of insertion failure.

A two piece suture anchor, including a rigid anchor body and a deformable cap that mates to the anchor body, may be used to decrease the risk of insertion failure and/or increase fixation in smaller anchors. When the deformable cap is pushed onto the rigid anchor body (or vice versa), the deformable cap expands radially outward, compressing into surrounding bone and further securing the suture anchor to bone. As such, the two piece suture anchors are sometimes referred to as “expanding anchors.” However, one of the problems with expanding anchors is that, once the cap is flexibly expanded, there is no way for the user to know if the expansion has been retained within the bone hole.

### SUMMARY

Described herein is an expanding suture anchor in which a distal end of the rigid anchor body includes at least one annular protrusion. A mating deformable cap includes at least one annular slot or ridge that engages the annular protrusion such that the annular slot and annular protrusion are coupled when the deformable cap is pushed or pulled onto the rigid anchor body. The engagement of the annular slot and the annular protrusion may lock the deformable cap and rigid anchor body into a mated position and inhibits distal retraction of the deformable cap from the anchor body. Advantageously, this ensures that once the suture anchor is expanded, it remains in its expanded state.

Further examples of the suture anchor of this disclosure may include one or more of the following, in any suitable combination.

In one example, the suture anchor of this disclosure includes a cannulated, rigid anchor body extending between a proximal end and a distal end, the distal end having a first portion distal to a second portion, and the first portion including a lateral protrusion. The suture anchor also includes a deformable cap including a cavity having a diameter larger than the first portion of the distal end of the anchor body and smaller than the second portion of the distal end of the anchor body, and at least one expansion slot formed through a lateral surface of the deformable cap or a similarly functioning ridge on the inner diameter of the cap. The at least one expansion slot or ridge is dimensioned to receive at least a portion of the lateral protrusion of the anchor body. Upon proximal advancement of the deformable cap with respect to the distal end of the anchor body, the deformable cap expands radially outward to accommodate at least the first portion of the distal end of the anchor body such that the lateral protrusion is received within the expansion slot and inhibits distal retraction of the deformable cap from the anchor body.

In further examples, the suture anchor of this disclosure has at least one transverse projection formed integrally with the anchor body. The anchor body is comprised of at least one of metals, polymers, bioabsorbable, and biocomposite materials. The deformable cap has a tapered distal end and is formed separately from the anchor body. The deformable cap is formed from PEEK, stainless steel, or Nitinol. The deformable cap is made of a material that is either the same as or different from a material of the anchor body. The at least one lateral projection can be a plurality of lateral projections and the at least one slot can be a plurality of slots. The deformable cap is attached to the anchor body by means of a suture routed through the deformable cap and the anchor body. The deformable cap further has at least one opening for the passage of a suture. The anchor body is comprised of at least one of metals, polymers, bioabsorbable and biocomposite materials.

These and other features and advantages will be apparent from a reading of the following detailed description and a review of the associated drawings. It is to be understood that both the foregoing general description and the following detailed description are explanatory only and are not restrictive of aspects as claimed.

### BRIEF DESCRIPTION OF THE DRAWINGS

The disclosure will be more fully understood by reference to the detailed description, in conjunction with the following figures, wherein:

FIG. 1 is an example of the suture anchor with expandable cap of this disclosure in transparent view;

FIG. 2 is an example of the suture anchor of FIG. 1 shown with a suture routed therethrough;

FIGS. 3A and 3B illustrate proximal movement of the expandable cap of FIG. 1 with respect to the distal end of the suture anchor; and

FIG. 3C is a detail illustration of the expandable cap of this disclosure.

### DETAILED DESCRIPTION

In the description that follows, like components have been given the same reference numerals, regardless of whether they are shown in different examples. To illustrate example(s) in a clear and concise manner, the drawings may not necessarily be to scale and certain features may be shown in somewhat schematic form. Features that are described and/or illustrated with respect to one example may be used in the same way or in a similar way in one or more other examples and/or in combination with or instead of the features of the other examples.

Comprise, include, and/or plural forms of each are open ended and include the listed parts and can include additional parts that are not listed. And/or is open ended and includes one or more of the listed parts and combinations of the listed parts.

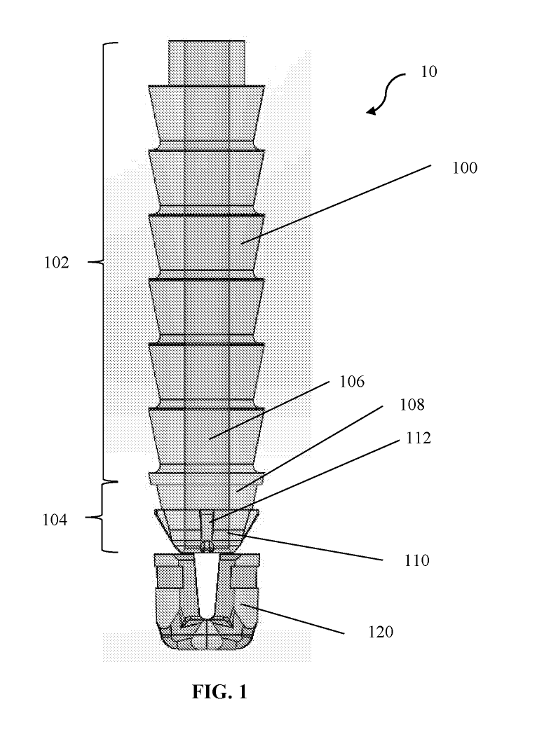

Referring now to FIG. 1, an example of a suture anchor 10 of this disclosure is illustrated in transparent view. The suture anchor 10 includes a cylindrical, rigid anchor body 100 having a proximal portion 102 and a distal portion 104. In FIG. 1, the anchor body 100 further comprises an open cannulation 106 extending the length of the anchor body 100, configured for the passage of at least one suture. However, it is contemplated by this disclosure that the anchor body 100 may be a solid, non-cannulated anchor, and/or may comprise a transverse eyelet through the anchor body 100 configured for the passage of a suture. The anchor body 100 may also include surface features along the length of the anchor body 100 in order to enhance fixation strength in bone. In FIG. 1, the surface features are shown as circumferential ribs, however other surface features, such as longitudinal ribs, barbs or the like, are also contemplated by this disclosure. The distal portion 104 of the anchor body 100 includes a proximal first portion 108 and a distal second portion 110. In examples, the second portion 110 has a diameter which is selected to be smaller than a diameter of the first portion 108, the purpose of which will be described in more detail below.

The anchor body 100 can be made from any combination of metals, polymers, bioabsorbable, or biocomposite material. For example, the anchor body 100 may be partially or entirely formed from a formulation of poly(lactic-co-glycolic) acid (PLGA), β-Tricalcium phosphate (β-TCP) and calcium sulfate, poly-L-lactic acid—hydroxyapatite (PLLA-HA), poly-D-lactide (PDLA), polyether ether ketone (PEEK) or variants thereof. Biocomposite examples made from a combination of PLGA, β-TCP, and calcium sulfate are absorbable by the body, which is beneficial to natural healing. An example formulation of PLGA, β-TCP, and calcium sulfate is described in U.S. Pat. No. 8,545,866, the entirety of which is herein incorporated by reference. A copolymer of polyglycolic acid (PGA) and polytrimethylene carbonate (TMC) is another example of a bioabsorbable material. Other commonly used materials that are capable of providing the strength needed to set the anchor body 100 into bone and to hold a suture and tissue in position while bone-to-tissue in-growth occurs are also contemplated by this disclosure.

Still referring to FIG. 1, it can be seen that a deformable cap 120 is disposed at the distal end of the anchor body 100. The cap 120 may be formed separately from and attached to the anchor body 100. For example, the cap 120 may be attached to the anchor body 100 by thin threads or other break-away features. The cap 120 is preferably comprised of a material which allows some flexibility, such as PEEK, and which may be the same material or a different material as the anchor body 100. It is also contemplated by this disclosure that metals, such as stainless steel or Nitinol, could also be used. In FIG. 1, the cap 120 is shown in as having a substantially square geometry, however, other suitable geometries are possible. The second portion 110 of the anchor body 100 may optionally include at least one transverse side boss 112 formed integrally with the anchor body 100 and forming an interference fit with the cap 120 for alignment and fixation of the cap 120 when the cap is inserted onto the anchor body 100. The side boss 112 may also prevent rotation of the cap 120 with respect to the anchor body 100.

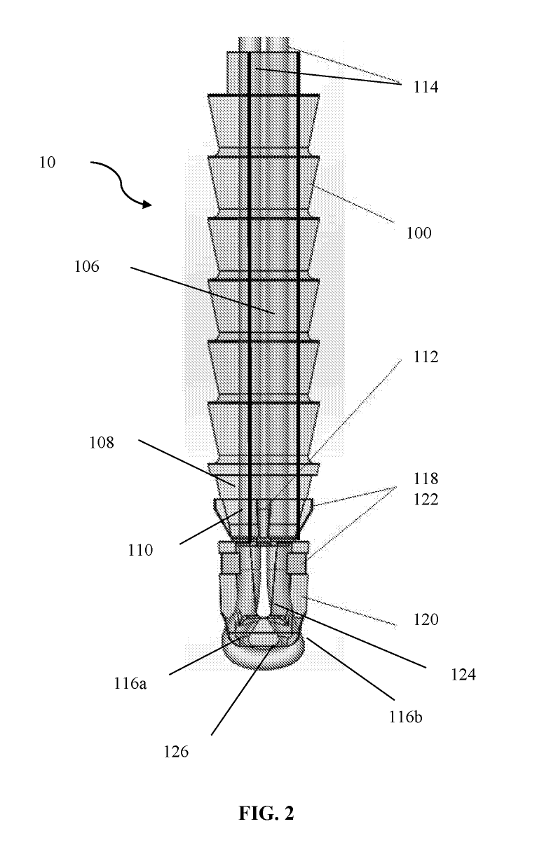

FIG. 2 shows another example of the anchor body 100 in transparent view. In FIG. 2, a suture 114 is shown doubled over and routed through the cannulation 106 of the anchor body 100. In this example, the suture 114 extends from the cannulation 106 outside of a first opening 116a in the cap 120 and back through a second opening 116b in the cap 120 to return to the cannulation 106. This configuration of the suture 114 not only holds the cap 120 onto the anchor body 100 but allows for slide in the suture 114 when one or both free ends of the suture 114 are pulled, which is advantageous for knot tying during the surgical procedure. However, knotless configurations of the anchor body 100 and suture 114, wherein the suture 114 is impinged within the anchor body 100 or the cap 120 to resist pull-out force on the suture 114, are also contemplated by this disclosure. In this case, the cap 120 may only include one opening for the passage of the suture 114. Additionally, solid configurations of the anchor body 100 and cap 120 are contemplated by this disclosure.

In FIG. 2, it can be seen that the second portion 110 comprises at least one lateral protrusion 118 formed integrally with the anchor body 100. The lateral protrusion 118 may extend radially from the anchor body 100. The cap 120 in turn comprises at least one slot 122 formed through a lateral surface of the cap 120 and configured to receive at least a portion of the at least one protrusion 118. In other examples, not shown, the cap 120 may have a similarly functioning ridge on the inner diameter of the cap 120. The cap 120 also includes an internal cavity 124. In examples, at least a portion of the internal cavity 124 has a diameter which is selected to be larger than a diameter of the second portion 110 of the anchor body 100 but smaller than a diameter of the first portion 108 of the anchor body 100. In examples the diameter of the internal cavity may be between about 1.5 mm and 5 mm. The cap 120 furthermore may include a tapered distal portion 126 adapted for insertion into bone.

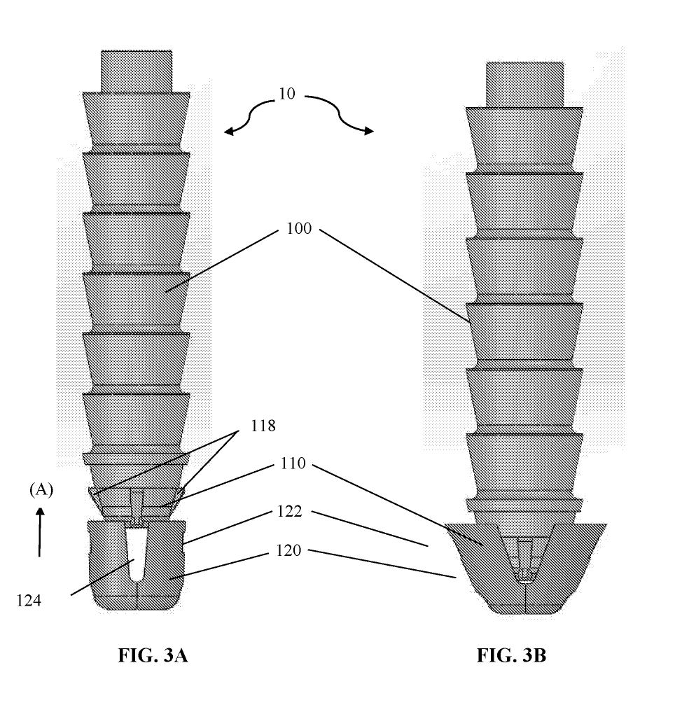

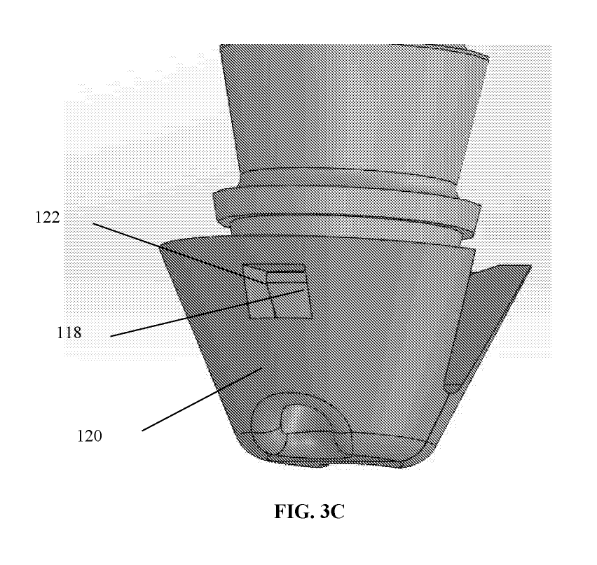

Turning now to FIGS. 3A and 3B, in use, the anchor body 100 may be inserted into a bone hole (not shown). For example, the entire anchor body 100 may be inserted into the bone hole by a pound-in force directed to the anchor body 100 through an inserter (not shown). Contact with the bone hole and/or other forces or mechanisms that can impart the relative motion of the cap 120 to the anchor body 100 causes proximal advancement (A) of the cap 120 with respect to the anchor body 100. In examples, the proximal advancement could be caused by tension applied to the suture 114 (FIG. 2). In yet further examples, the anchor body 100 could be slidably mounted to an inner shaft of the inserter while the cap 120 is disposed on the distal end of the inner shaft. The proximal advancement (A) is thus caused by the inner shaft retracting through the anchor body 100 with respect to the outer shaft, bringing the cap 120 in contact with the anchor body 100. The force of the proximal advancement (A) causes the second portion 110 to enter the internal cavity 124 of the cap 120. This in turn causes the cap 120 to expand radially outward to accommodate at least the second portion 110 of the anchor body 100. The distal end of the anchor body 100, meanwhile, exhibits little to no deformation. Concurrently, the at least one lateral protrusion 118 is received within the at least one slot 122 and inhibits distal retraction of the cap 120 from the anchor body 100. In examples, the cap 120 is also locked into place relative to the anchor body 100. This increases fixation strength between the anchor body 100 and surrounding bone.

FIG. 3C is a detailed view of the cap 120, showing the at least one lateral protrusion 118 received within the at least one slot 122. In FIG. 3C, the slot 122 is shown as having a substantially square geometry. However, other suitable geometries are contemplated by this disclosure. For example, the slot 122 may be trapezoidal or triangular.

In examples, not shown, the expanding portion of the suture anchor 10 may be the distal portion 104 of the anchor body 100. For example, a proximal force may cause the deformable cap 126 to enter into an opening at the distal portion 104 of the anchor body 100, causing outward expansion of the distal portion 104. The locking features may then comprise internal locking features rather than external locking features (i.e., the protrusions may be located on the inside of the opening of the distal portion 104 to mate with the slots 122 on the surface of the cap 120). In other examples, a diameter of the second portion 110 of the anchor body 100 may be larger than a diameter of the first portion 108, such that the cap 120 is configured to snap into a recess formed by the first portion 108.

The suture anchor 10 of this disclosure is a micro anchor sized appropriately for, e.g., instability or rotator cuff repair. However, suture anchor 10 of this disclosure could also be adapted or scaled for other types of surgical repair.

While this disclosure has been particularly shown and described with references to preferred embodiments thereof, it will be understood by those skilled in the art that various changes in form and details may be made therein without departing from the spirit and scope of the present application as defined by the appended claims. Such variations are intended to be covered by the scope of this present application. As such, the foregoing description of embodiments of the present application is not intended to be limiting, the full scope rather being conveyed by the appended claims.

1. A suture anchor comprising:
a cannulated, rigid anchor body comprising a proximal portion, a distal portion, and a longitudinal axis extending between the proximal and distal portions, the distal portion further comprising a second portion distal to a first portion, the second portion including at least one lateral protrusion; and
a deformable cap comprising:
a cavity having a diameter larger than a diameter of the second portion of the distal end of the anchor body and smaller than a diameter of the first portion of the distal end of the anchor body; and
at least one slot formed through a lateral surface of the deformable cap, the at least one slot dimensioned to receive at least a portion of the at least one lateral protrusion of the anchor body;
wherein, upon proximal advancement of the deformable cap with respect to the distal end of the anchor body, the deformable cap expands radially outward to accommodate at least the second portion of the distal end of the anchor body such that the at least one lateral protrusion is received within the at least one slot and inhibits distal retraction of the deformable cap from the anchor body.

2. The suture anchor of claim 1, wherein the anchor body comprises at least one transverse projection formed integrally with the anchor body.

3. The suture anchor of claim 1, wherein the deformable cap comprises a tapered distal end.

4. The suture anchor of claim 1, wherein the deformable cap is formed separately from the anchor body.

5. The suture anchor of claim 1, wherein the deformable cap is comprised of PEEK.

6. The suture anchor of claim 1, wherein the deformable cap is comprised of stainless steel or Nitinol.

7. The suture anchor of claim 1, wherein the deformable cap is comprised of a material that is different from a material of the anchor body.

8. The suture anchor of claim 1, wherein the deformable cap is comprised of a material that is the same as a material of the anchor body.

9. The suture anchor of claim 1, wherein the at least one lateral projection is a plurality of lateral projections.

10. The suture anchor of claim 1, wherein the at least one slot is a plurality of slots.

11. The suture anchor of claim 1, wherein the deformable cap is attached to the anchor body by means of a suture routed through the deformable cap and the anchor body.

12. The suture anchor of claim 1, wherein the deformable cap further comprises at least one opening for the passage of a suture.

13. The suture anchor of claim 1, wherein the anchor body is comprised of at least one of metals, polymers, bioabsorbable, and biocomposite materials.

14. The suture anchor of claim 1, wherein a surface of the second portion comprises at least one transverse side boss for alignment and fixation of the cap.

15. The suture anchor of claim 1, wherein the cap is locked on the distal portion of the anchor body.

16. The suture anchor of claim 1, wherein a diameter of the cavity is between about 1.5 mm and 5 mm.
